(19) United States
(12) Patent Application Publication
(10) Pub. No.: US 2024/0268805 A1
(43) Pub. Date: Aug. 15, 2024

(54) ACCESS INTO INTERVENTRICULAR SEPTUM USING A PUNCTURE CATHETER DEVICE

(71) Applicants: TAU MEDICAL INC., Centreville, VA (US); TAU MEDICAL INC., Busan (KR)

(72) Inventor: June-Hong Kim, Busan (KR)

(21) Appl. No.: 18/568,537

(22) PCT Filed: Jun. 13, 2022
(86) PCT No.: PCT/US22/33320

§ 371 Date: Dec. 8, 2023

Related U.S. Application Data
(60) Provisional application No. 63/209,962, filed on Jun. 12, 2021.
(60) Provisional application No. 63/221,813, filed on Jul. 14, 2021.

(51) Int. Cl. A61B 17/00; A61B 17/34; A61B 90/00

(52) U.S. Cl. CPC A61B 17/00234; A61B 17/34; A61B 90/39; A61B 2017/00017; A61B 2017/00247; A61B 2017/00305; A61B 2017/00323; A61B 2017/00331; A61B 2017/00367; A61B 2090/3966

(57) ABSTRACT

A method of creating a transvenous access pathway into an interventricular septum of a patient's heart. This access pathway could be used for cardiac procedures such as cardiac pacing, RF (radiofrequency) ablation, or mitral valve cerclage procedures. The method uses a catheter device that is steerable at its distal end. After passing through the coronary sinus and into coronary vein (e.g. great cardiac vein), a puncture tool is advanced through the catheter device and made to puncture through the vein wall. To facilitate this, the distal end of the catheter device may be bent at an angle towards the vein wall. The puncture tool is pushed to bore through the myocardium towards the interventricular septum. This creates an entry passageway from the coronary vein, through the myocardium, and into the interventricular septum. This entry passageway could be used for insertion of an electrode lead or a mitral loop cerclage wire.

### TECHNICAL FIELD

This invention relates to catheter devices for performing transvascular cardiac procedures.

### BACKGROUND

Recent developments in interventional cardiology have led to a need for creating a transvenous access pathway into an interventricular septum of a patient's heart. For example, this pathway could be used for novel catheter-based treatments for mitral valve regurgitation by a mitral loop cerclage, physiologic pacing, or septal reduction by intra-septal radiofrequency (RF) ablation for hypertrophic cardiomyopathy. This approach is relatively easier if the patient has a septal perforating vein into the interventricular septum. However, if the patient does not have a suitable septal perforating vein into the interventricular septum, this approach is more difficult. Thus, there is a need for a more general technique for creating a transvenous access pathway that could be used in patients with our without a suitable septal perforating vein.

### SUMMARY

This invention is for creating a transvenous access pathway into an interventricular septum of a patient's heart. There are a variety of different types of cardiac procedures for which such an access pathway could be useful, such as cardiac pacing, RF (radiofrequency) ablation, or mitral valve cerclage procedures. These are described in more detail below. The method uses a catheter device that is steerable at its distal end. An example of a catheter device that could be used include those described in patent application publication WO 2021/119636 (Tau Cardio, Inc.) titled “Septal Cross System”, which is incorporated by reference herein. Other examples of catheter devices that could be used are further described herein.

The catheter device comprises a main tube, which comprises a distal end segment, a distal tip, and a lumen. As used herein, ‘distal end segment’ means the portion of the main tube encompassing from the distal tip of the main tube to a distance of 5 cm inward (proximal) from the distal tip. The length of the main tube could be in the range of 60-180 cm long. The catheter device further comprises a puncture tool that travels through the lumen. The puncture tool could be inserted into the lumen of the catheter device at any suitable step in the cardiac procedure.

The main tube is inserted into an entry vein (such as the femoral, subclavian, or jugular vein). The main tube is advanced further into the vena cava (superior or inferior). The main tube is advanced into the right atrium of the heart. From there, the main tube is inserted into the coronary sinus and advanced through a coronary vein. Examples of coronary veins that could be traversed include tributaries that drain into the coronary sinus, such as the great cardiac vein, left ventricle vein, middle cardiac vein, left marginal vein, and septal perforating veins.

The distal tip is positioned at a target site within the coronary vein that is near the interventricular septum (e.g. within a distance of 4.0 cm). The puncture tool is introduced into the catheter device so that it travels through the main tube via a lumen of the main tube. The puncture tool could be any type of wire-like instrument that is capable of boring through the vein wall and into the myocardium. For example, the puncture tool could be a wire, drill tip, sharp needle point, etc.

The puncture tool is advanced through the lumen of the main tube in a distal direction along the path of the main tube (i.e. through the vena cava, right atrium, coronary sinus, and coronary vein). The puncture tool is advanced out of the distal tip opening of the main tube and is pushed to puncture through the vein wall. The puncture tool is further advanced into the myocardium towards the interventricular septum. This creates an entry passageway from the coronary vein, through the myocardium, and into the interventricular septum.

Any suitable amount of force may be used to puncture through the vein wall. In situations where the patient has a septal perforating vein and the distal tip of the main tube is positioned therein, relatively less force may be needed. For example, the amount of push force applied could be less than 0.20 N (newtons) to puncture through the vein wall. In other situations (e.g. where the patient does not have a septal perforating vein), relatively more push force may be needed. For example, the amount of push force applied could be in the range of 0.20-1.25 N (newtons) to puncture through the vein wall. This amount of force may be suitable in situations where the distal tip of the main tube is in the great cardiac vein, and puncture of the wall therethrough is required. For applying the pushing force, using the opposing wall of the coronary vein may be used as a buttress support at the distal end segment of the main tube.

In some embodiments, at the target site for puncturing the vein wall, the distal end segment of the main tube is bent at an angle relative to the longitudinal axis of the main tube. As such, the distal tip opening is pointed at an angle towards the vein wall. For example, this angle could be at least 45° relative to the longitudinal axis of the main tube. With this bend in the main tube, the puncture tool is advanced towards the vein wall at an angle and made to puncture therethrough.

In some embodiments, the main tube comprises a bendable segment at the distal end segment thereof. The bendable segment can be made to bend by operator control (e.g. like a steerable tip). In some cases, the bendable segment is more flexible than another segment of the main tube that is proximal to the bendable segment (e.g. the bendable segment is more flexible than other parts of the main tube). In some cases, the main tube comprises a coil element at the bendable segment thereof. Having a coil element makes the bendable segment have a spring-like compliance that improves the steering ability of the catheter device. In some cases, the length of the bendable segment is in the range of 10-40 mm long.

In some embodiments, the main tube comprises a pre-formed C-shape curve segment that extends from a proximal point A to a distal point B. Point A is located within an interval that is 6.0-15 cm of the distal tip of the main tube. Point B is located within an interval that is 0.4-4.0 cm from the distal tip of the main tube. Point B is located distal to point A. The length of the C-shape curve segment (distance from point A to point B) could be in the range of 5-20 cm long. In some cases, the steering wire is located on the inside curvature of the C-shape curve. In use, the main tube is inserted into the coronary sinus so that the C-shape curve of the main tube follows the curve of the coronary sinus. This pre-formed C-shape curve could help in setting the main tube at a good angle towards the coronary sinus and giving torque-ability through the coronary vein.

In some embodiments, the catheter device further comprises a steering wire that operates to cause bending of the main tube. The steering wire is fixed to the main tube at the distal end segment thereof. Bending is actuated by pushing or pulling the steering wire. Straightening or bending may be achieved by pushing or pulling, depending on the specific configuration of the catheter device. The distal end of the steering wire is affixed to the main tube near its distal tip. In some cases, the distal end of the steering wire is affixed to the main tube at a location on the main tube that is within 1.5 cm from the distal tip of the main tube. The length of the steering wire could be in the range of 80-200 cm long. The length of the steering wire may be shorter than the length of the puncture tool.

In some embodiments, the catheter device further comprises a handle assembly, which comprises a steering actuator. The steering wire is coupled to the steering actuator which acts to push or pull the steering wire. The steering actuator may have any suitable components to perform this action, such as knobs, dials, sliders, levers, etc.

In some embodiments, the main tube further comprises a second lumen that is separate from the (first) lumen. The steering wire travels through the separate second lumen of main tube. In some cases, the main tube comprises an exit hole out of the second lumen. The steering wire exits the second lumen out of the exit hole. This exit hole could be located at a point that is within 4-40 mm of the distal tip of the main tube.

In some embodiments, the distal end segment of the main tube comprises two or more radiopaque markers. The distance between the markers could be in the range of 2-8 mm. In some cases, the main tube comprises at least three radiopaque markers. This could be useful in allowing the operator to assess the amount of bending by imaging under x-ray fluoroscopy. The amount of deviation that the radiopaque markers are from straight alignment would indicate the degree of bending.

In some embodiments, the catheter device is used to perform a venogram of the heart. With the distal tip of the main tube inside the coronary sinus or further into a coronary vein, the contrast agent is injected through the catheter device and into the coronary vein. In some cases, a pressurized venogram is performed by putting a blockage at or near the (within 1.5 cm) of the coronary sinus entrance. This allows for a pressurized venogram to be performed to allow better visualization of smaller distal tributary veins of the coronary sinus.

In some embodiments, the method is used for a inserting an electrode lead into the patient's heart. Examples of electrode leads include those used for cardiac pacing or RF (radiofrequency) ablation. The method comprises introducing the electrode lead into the catheter device so that it travels the same path along the main tube, and then through the entry passageway in the myocardium, and into the interventricular septum. The electrode of the electrode lead is positioned inside the interventricular septum. In situations where the same lumen is used for advancing the electrode lead, the puncture tool is withdrawn out of the catheter device, and the electrode lead is advanced through this same lumen of the main tube.

Cardiac pacing in the interventricular septum may be useful for treating dysrhythmias. For example, the treatment could be para-Hisian pacing of the right ventricular close to the His bundle or proximal right bundle branch (RBB). This may be useful in patients who have atrioventricular (AV) block as a cause of their dysrhythmia. RF (radiofrequency) ablation in the interventricular septum may be useful for treating various cardiac disease conditions, such as hypertrophic cardiomyopathy.

In some embodiments, the method is used for a mitral valve cerclage procedure. Examples of such procedures are described in Kim et al, “Mitral cerclage annuloplasty, a novel transcatheter treatment for secondary mitral valve regurgitation: Initial results in swine” (2009) J Am Coll Cardiol. 54(7): 638-651; and Park et al, “Mitral Loop Cerclage Annuloplasty for Secondary Mitral Regurgitation: First Human Results” (2017) JACC Cardiovasc Interv. 10(6): 597-610. These references are incorporated by reference herein.

The method comprises introducing the mitral loop cerclage wire into the catheter device so that it travels the same path along the main tube, and then through the entry passageway in the myocardium, and into the interventricular septum. The mitral loop cerclage wire may then exit out of the interventricular septum to form the desired loop. In situations where the same lumen is used for advancing the cerclage wire, the puncture tool is withdrawn out of the catheter device, and the cerclage wire is advanced through this same lumen of the main tube.

### BRIEF DESCRIPTION OF THE DRAWINGS

FIG. 1 shows an overall view of a catheter device.

FIG. 2 shows a close-up side view at the distal portion of the catheter device.

FIG. 3 shows a close-up cross-section view at the distal portion of the catheter device.

FIGS. 4-6 show the bending operation of the catheter device. FIG. 4 shows the twist knob in idle position with no tension being applied to the steering wire. FIG. 5 shows the slider moving backwards. FIG. 6 shows the distal tip of the main tube being deflected at an angle.

FIGS. 7 and 8 show close-up views of the bendable segment. In FIG. 7, the bendable segment is bent at 90° angle. In FIG. 8, the bendable segment is further flexed to bend at 135° angle.

FIGS. 9-11 show an example of how the steering mechanism could work. FIG. 9 shows the main tube inside the coronary vein. FIG. 10 shows the puncture tool inside the main tube, which is in bent configuration. FIG. 11 shows further bending of the main tube for aiming towards the myocardium at a steeper angle.

FIG. 12 shows a transverse cross-section of the heart and demonstrates an example of the overall insertion procedure for the catheter device.

FIG. 13 shows how an external ultrasound probe could be used by the operator to help guide the puncture procedure.

FIGS. 14-16 show how the bendable segment of the main tube is made to bend by retraction of the steering wire. FIG. 14 shows the main tube in a relaxed pose (no tension applied). FIG. 15 shows bending of the main tube caused by retraction of the steering wire. FIG. 16 shows further bending of the main tube with further retraction of the steering wire.

FIG. 17 shows an example of a conventional cardiac pacemaker device that could be implanted using the catheter device.

### DETAILED DESCRIPTION OF EXAMPLE EMBODIMENTS

To assist in understanding the invention, reference is made to the accompanying drawings to show by way of illustration specific embodiments in which the invention may be practiced. The drawings herein are not necessarily made to scale or actual proportions. For example, lengths and widths of the components may be adjusted to accommodate the page size.

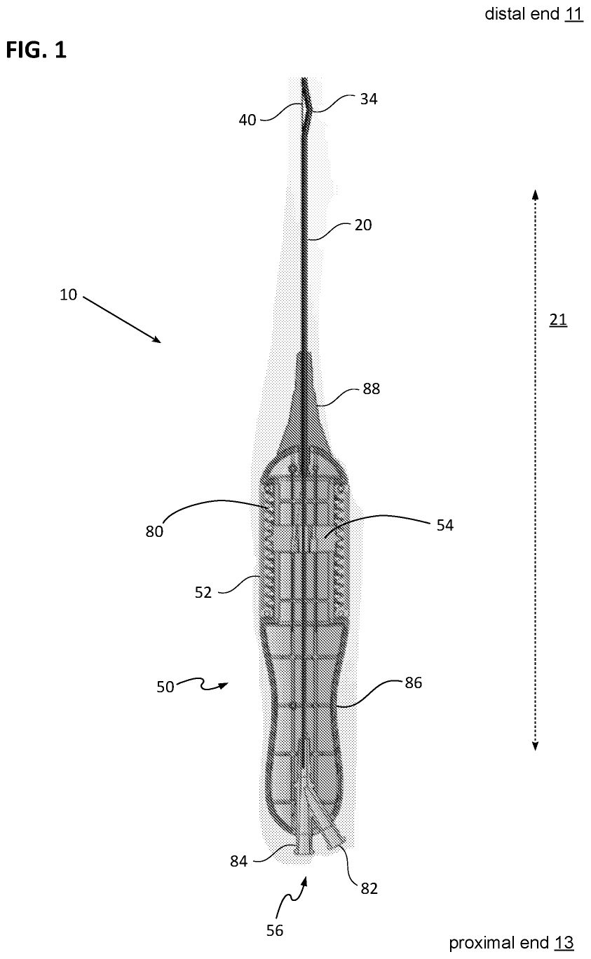

FIG. 1 shows an example of a catheter device 10 for creating a transvenous access pathway into an interventricular septum in a patient's heart. The proximal end 13 and distal end 11 directions relative to the catheter device 10 are indicated. Towards the distal end 11, the catheter device 10 comprises a flexible main tube 20 which has multiple lumens (two or more) therein. This configuration defines the longitudinal axis 21 of the main tube 20 (straight vertical in this drawing). The main tube 20 comprises a bendable segment 34. The catheter device 10 also comprises a steering wire 40 for manipulating the bendable segment 34 of the main tube 20.

Towards the proximal end 13, the catheter device 10 comprises a handle assembly 50, which comprises a cone-shaped hood 88 where the main tube 20 attaches thereto. The handle assembly 50 further comprises a handle grip 86. At the back of the handle assembly 50, there is a hub 56 with a main port 84 and an accessory port 82. A guidewire and puncture tool (not shown here) is inserted into main port 84, which is the opening for the primary lumen 26 (not shown here) of the main tube 20. The handle assembly 50 comprises a twist knob 52 and a slider 54. There is a spiral railing 80 on the inside of twist knob 52. The twist knob 52 engages with the slider 54 on the spiral railing 80. With this configuration, turning the twist knob 52 (clockwise or counterclockwise) causes the slider 54 to move forward or backwards as driven by the spiral railing 80. As explained in more detail below, this causes bending at the bendable segment 34 of the main tube 20.

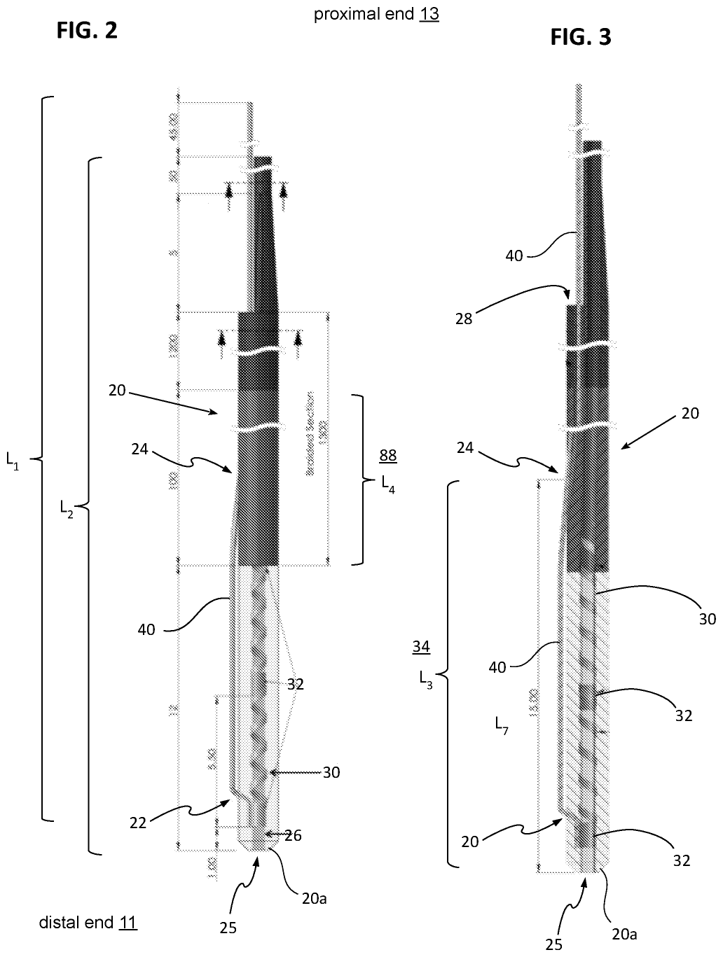

FIGS. 2A and 2B show close-up views at the distal portion of the catheter device 10. Shown here, the main tube 20 has a distal tip 20a with an opening 25 thereon. The opening 25 is part of a primary lumen 26 through which the puncture tool 44 (not shown here) travels. An electrode lead or cerclage wire could be inserted through this same primary lumen 26 used by the puncture tool 44 (after it has been withdrawn). In addition to the primary lumen 26 for the puncture tool 44 (not shown), the main tube 20 has a secondary lumen 28 for the steering wire 40. Located at the secondary lumen 28, is a proximal side hole 24, which serves as an exit hole from the secondary lumen 28.

The steering wire 40 exits the secondary lumen 28 out through the proximal side hole 24 and travels alongside and external to the body of the main tube 20. The steering wire 40 reenters the main body 20 through distal side hole 22. Within the main tube 20, the distal end of steering wire 40 is embedded therein. In this way, the distal end of steering wire 40 is attached to near the distal tip 20a of the main tube 20. A distal hole 22 is not necessary for attachment of the steering wire 40 to the main tube 20. For example, in other embodiments, the distal end of the steering wire 40 could be attached to the outer surface of the main tube 20 at or near its distal tip 20a.

FIGS. 2 and 3 also show some of the relevant dimensions of catheter device 10. The total length L2 of the main tube 20 is about 120 cm long. The length L3 of bendable segment 34 is about 18 mm long. The length L1 of the steering wire 40 is longer than the length L2 of the main tube 20. Here, the length L1 of steering wire 40 is about 150 cm long. The portion of steering wire 40 that travels outside the body of the main tube 20 (between side holes 22 and 24) is about 12 mm long (when then main tube 20 is in straightened configuration). The distance from the proximal side hole 24 to the distal tip 20a of the main tube 20 is about 15 mm. The length of the secondary lumen 28 (for the steering wire 40) is shorter than the length of the primary lumen 26 for the puncture tool 44. These figures also show a metal coil 30 within the bendable segment 34 of the main tube 20. The metal coil 30 provides elastic flexibility to the bendable segment; that is, allowing the bendable segment 34 to be elastically deformable. These figures also show three radiopaque markers 32.

The main tube 20 also has a pre-formed C-shape curve segment 88. That is, the main tube 20 at this curve segment 88 has a bias to form C-shape. This curve segment 88 encompasses a portion of the main tube 20 from the proximal end of the bendable segment 34 to a point further proximal thereof. In this example, the C-shape curve segment 88 has a length L4 of about 10 cm. This C-shape curve is compatible with the natural curving path of the coronary sinus. Having this C-shape curve may be useful in ensuring that the main shaft 20 is oriented so that bending at the bendable segment 34 correctly points the distal tip inward towards the interventricular septum when the vein puncture is performed.

The main tube 20 has three radiopaque marker bands 32 that are 1.0 mm wide for imaging under x-ray fluoroscopy. This allows the operator to assess the position of the distal end portion of the main tube 20. Here, the marker bands 32 are spaced at a distance of about 5 mm apart. Having multiple marker bands 32 in spaced intervals allows the operator to also assess the shape of the distal end portion of the main tube 20, i.e. amount of bending thereof. For example, a straight alignment of the three marker bands 32 would indicate no bending at the distal end portion of the main tube 20. However, the three marker bands 32 being out of straight alignment would give the operator an estimate of the amount of bending.

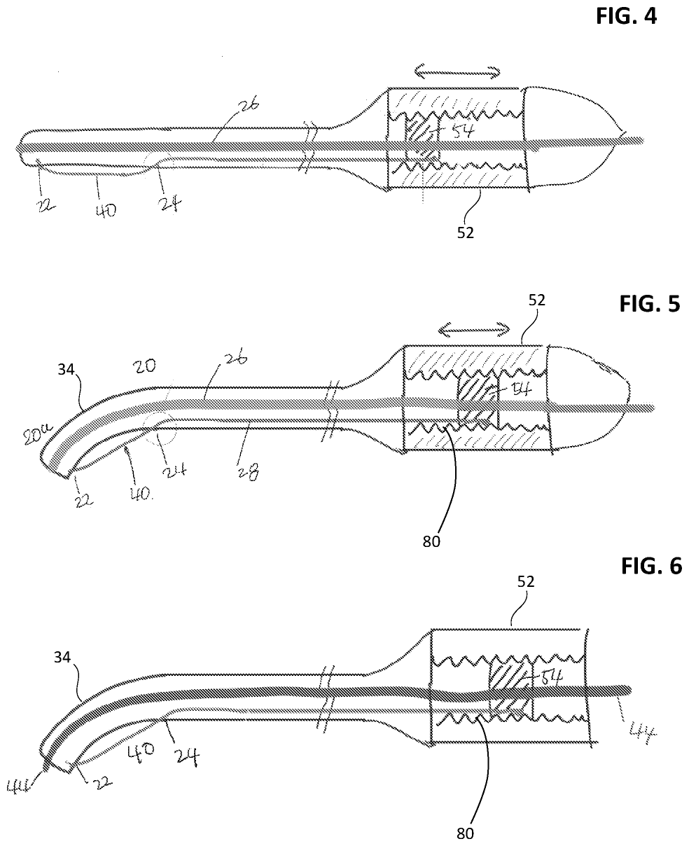

FIGS. 4-6 show the bending operation of the catheter device 10. Shown here is the main tube 20 and the primary lumen 26 contained therein. Also shown is a steering wire 40 that travels through the separate secondary lumen 28 of the main tube. The steering wire 40 exits the secondary lumen 28 through the proximal side hole 24, and is fixed to the main tube 20 at the distal side hole 22. The twist knob 52 holds the slider 54, which engages with the spiral railing 80 on the inside of twist knob 52. Rotating the twist knob 52 causes the slider 54 to move back and forth. The proximal end of the steering wire 40 is attached to the slider 54. This actuation mechanism causes the steering wire 40 to be pushed or pulled by turning of twist knob 52. This is the steering control mechanism for the catheter device 10.

In FIG. 4, the twist knob 52 is in idle position with no tension being applied to the steering wire 40. The main tube 20 is in a relaxed configuration with no forced bending at the distal end portion. In FIG. 5, the twist knob 52 is twisted by the operator, causing slider 54 to slide backwards along railing 80. This causes slider 54 to pull the steering wire 40 backward. Because of the pull tension applied to the steering wire 40, the distal end of the steering wire 40 pulls back the distal tip 20a of the main tube 20. This bending causes the bendable segment 34 of the main tube 20 to form a C-shape curve, pointing the distal tip 20a in a direction away from the longitudinal axis of the main tube 20.

In FIG. 6, with the distal tip 20a deflected at an angle, the puncture tool 44 is advanced forward so that it exits out of the opening at distal tip 20a of the main tube 20. Because of the C-shape bend at the bendable segment 34 of the main tube 20, the puncture tool 44 is pointed at an angle relative to the longitudinal axis of the main tube 20. As seen here, the length of the steering wire 40 is shorter than the length of the puncture tool 44.

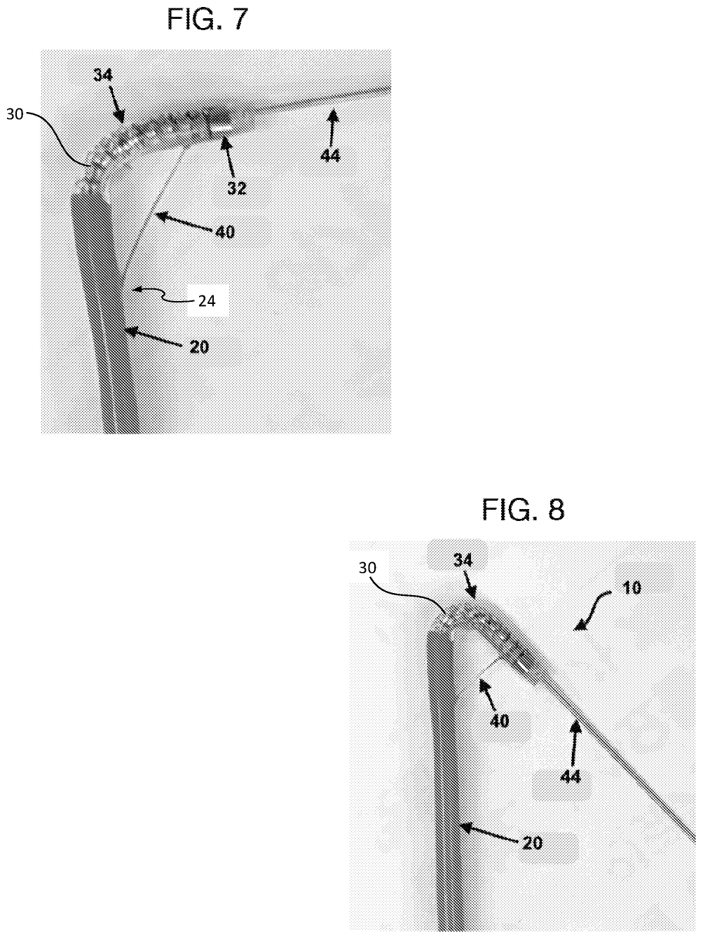

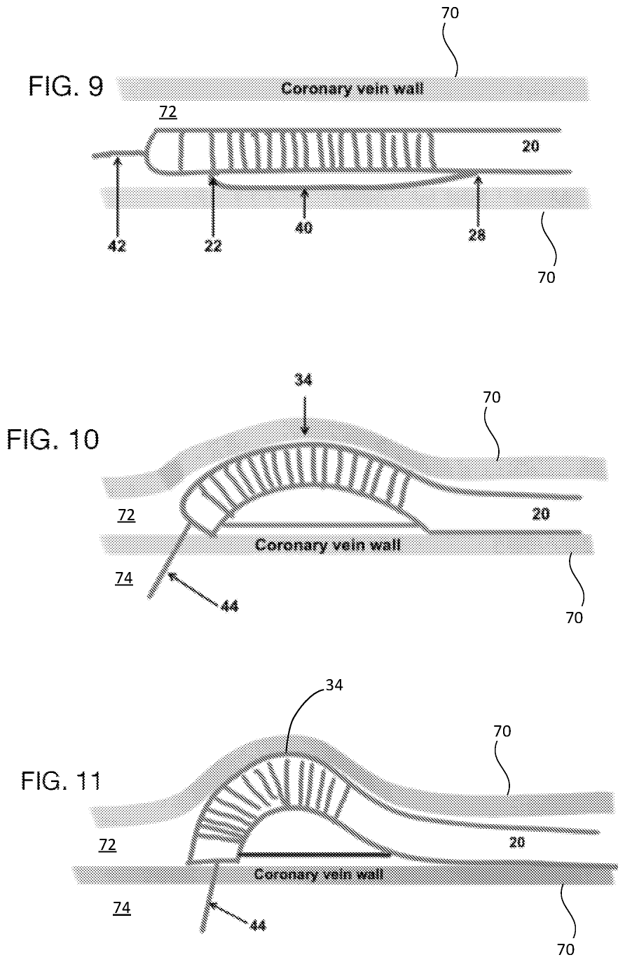

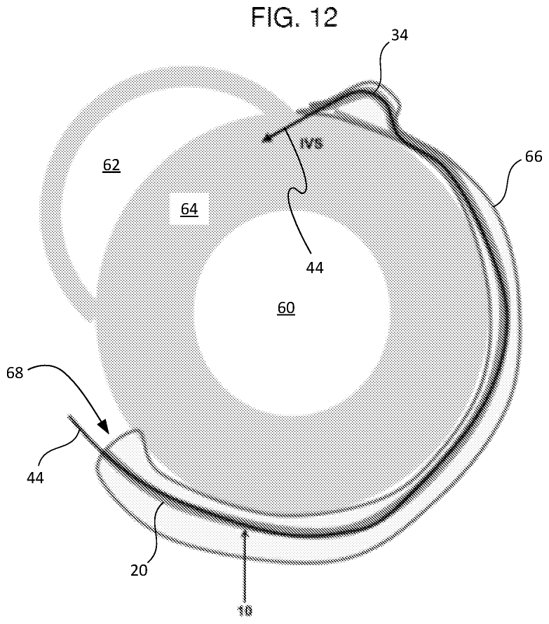

FIGS. 7 and 8 show close-up views of the bendable segment 34. The main tube 20 comprises a coil element 30 at the bendable segment 34. This coil element 30 facilitates steering by functioning as a spring-like counteraction. This coil element 30 could be embedded within the wall of the main tube 20 or could be a sheath around the primary lumen 26 (not shown here). The coil element 30 wraps around the primary lumen 26 in a spiral configuration and is coaxial thereto (common axis), which could serve to reinforce the primary lumen 26. The steering wire 40 exits out of the main tube 20 at side hole 24 and travels outside the body of main tube 20. The steering wire 40 then attaches to the main tube 20 at a point further distal.

In FIG. 7, the bendable segment 34 is bent at a 90° angle relative to the initial zero degree starting point when the main tube 20 is in straightened configuration (i.e. relative to the longitudinal axis). In FIG. 8, the bendable segment 34 is further flexed to bend at a 135° angle from the initial zero degree starting point. As seen herein, the puncture tool 44 is pointed at different angles caused by the bending of bendable segment 34. Also shown here is the distal-most of the three radiopaque markers 32. As seen here, the coil element 30 is located distal to the side hole 24 where steering wire 40 exits the main tube 20. The puncture tool 44 in assembled combination with the catheter device 10 could be considered a septal puncture assembly as another aspect of the invention.

FIGS. 9-11 show how the steering mechanism could point the puncture tool 44 in an angled direction. FIG. 9 shows the main tube 20 inside the coronary vein 72 that is enclosed by vein walls 70. The main tube 20 is positioned so that its distal tip is positioned near the site where the vein wall 70 will be punctured. Also shown is the guidewire 42 which is used to bring the main tube 20 to this target location within the coronary vein 72. The steering wire 40 is in a relaxed state and the distal end portion of the main tube 20 has a straight configuration.

In FIG. 10, the guidewire 42 (not shown) has been withdrawn from the primary lumen 26 (not shown) and replaced with insertion of the puncture tool 44 into the main tube 20 via the same primary lumen 26. To perform the puncture procedure, the steering wire 40 is retracted backwards (proximal) in a tension state. Because the distal tip of the steering wire 40 is affixed to the main tube 20 at the side hole 22, this backwards pulling of the steering wire 40 causes the main tube 20 to bend at the bendable segment 34. This bending causes this distal tip of the main tube 20 to point at an angle more towards the direction of the vein wall 70. With this bent configuration of main tube 20, the puncture tool 44 is advanced forward with the force necessary to puncture through the vein wall 70 and into the myocardium 74. Notice that this causes a bulging out at the opposing wall 70 of the coronary vein 72 where the bendable segment 34 presses against the opposing wall 70.

In FIG. 11, the steering wire 40 is retracted further for more tension. This causes the bendable portion 34 to bend further so that main tube 20 and puncture tool 44 are aimed at a steeper angle towards the myocardium 74. The puncture tool 44 is advanced forward into the myocardium 74 to create an entry passageway into the myocardium 74 towards the interventricular septum. This passageway will be used by the electrode lead to be inserted afterwards.

FIG. 12 shows the situation in FIG. 11 in a transverse cross-section of the heart and demonstrates the overall insertion procedure for the catheter device 10. In this view, the right ventricle 62 and the left ventricle 60 are seen in transverse cross-section view. For establishing the insertion pathway, a guidewire (not shown here) is inserted into the coronary sinus 68 located within the right atrium of the heart. From there, the guidewire is advanced through the great cardiac vein. The catheter device 10 is made to follow along this path by inserting the proximal end of the guidewire into the primary lumen of the main tube 20. The catheter device 10 is then advanced forward along the guidewire so that main tube 20 is inside the great cardiac vein 66. As such, the catheter device 10 is inserted into the coronary sinus 68 and through the great cardiac vein 66. The catheter device 10 is advanced so that the distal tip of the main tube 20 is positioned close to the interventricular septum (IVS) 64.

In this approach to the interventricular septum 64, the main tube 20 may travel through other coronary veins such as a septal perforator vein. However, not all people have a septal perforator vein, and this invention does not require the presence of such. With the catheter device 10 in position at the target site, the guidewire is then exchanged with the puncture tool 44. The bendable portion 34 of the main tube 20 is made to bend. The puncture tool 44 is advanced forward so that it punctures through the cardiac vein 66 at an angle and penetrates into the myocardium towards the interventricular septum 64.

The puncture tool 44 creates an entry passageway into the myocardium to the interventricular septum 64. With this entry passageway established, the puncture tool 44 is exchanged for the electrode lead or cerclage wire (not shown here). In the case of an electrode lead, it is advanced so that its electrode is positioned inside the interventricular septum 64. The electrode lead is thus implanted in the interventricular septum 64 and can be activated (e.g. for cardiac pacing or ablation treatment).

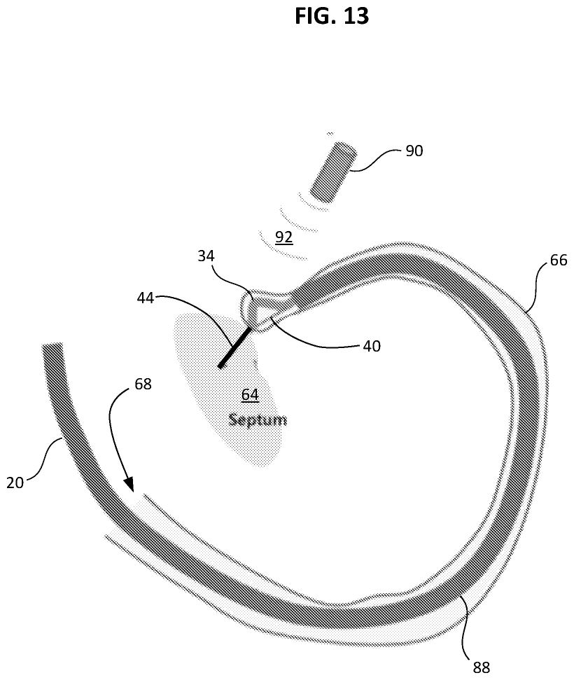

FIG. 13 shows how an external ultrasound probe 90 could be used by the operator to help guide the puncture procedure. The ultrasound probe 90 emits acoustic waves 92 that provide imaging of the distal end portion of the main tube 20. The puncture tool 44 may also be visible in the ultrasound image. Also shown here is the C-shape curve segment 88 of the main tube 20. This curve segment 88 conforms with the natural curving path of the coronary sinus 68. This ensures that main tube 20 is oriented correctly so that bending segment 34 bends in the outward direction so that the deflection angle points towards the interventricular septum 64.

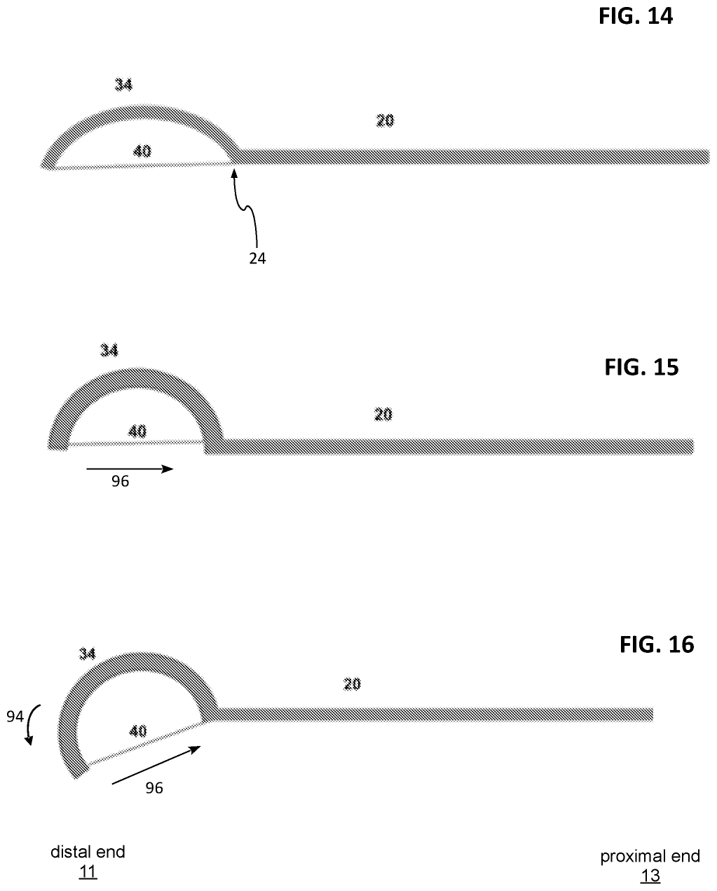

FIGS. 14-16 show how the bendable segment 34 of the main tube 20 is made to bend by retraction of the steering wire 40. In FIG. 14, the steering wire 40 exits out of the main tube 20 via proximal side hole 24 and is in a relaxed pose (no tension applied). In FIG. 15, the steering wire 40 is retracted backwards (proximal) in the direction of arrow 96. Because the distal tip of the steering wire 40 is affixed to the main tube 20 at the distal side hole 22, this backwards pulling of the steering wire 40 causes the main tube 20 to bend at the bendable portion 34. In FIG. 16, the steering wire 40 is retracted further for more tension. This causes the bendable portion 34 to bend further and aim the main tube 20 and puncture tool 44 at a steeper angle.

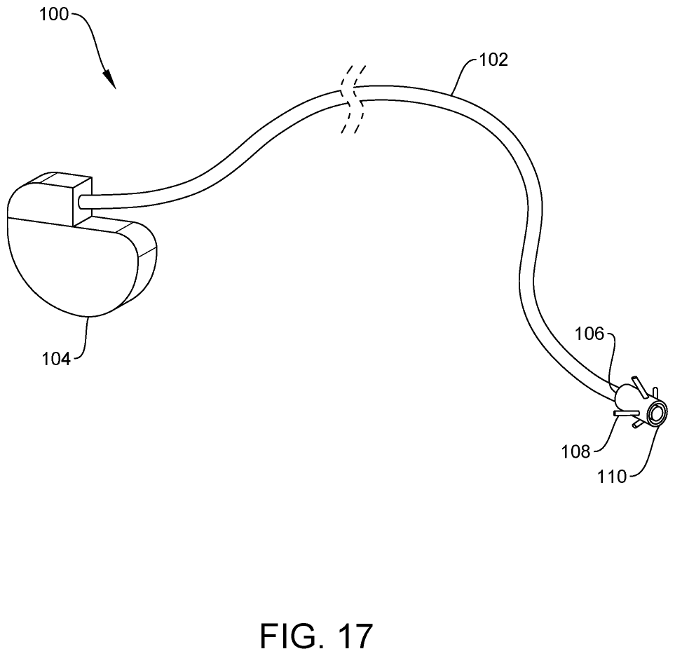

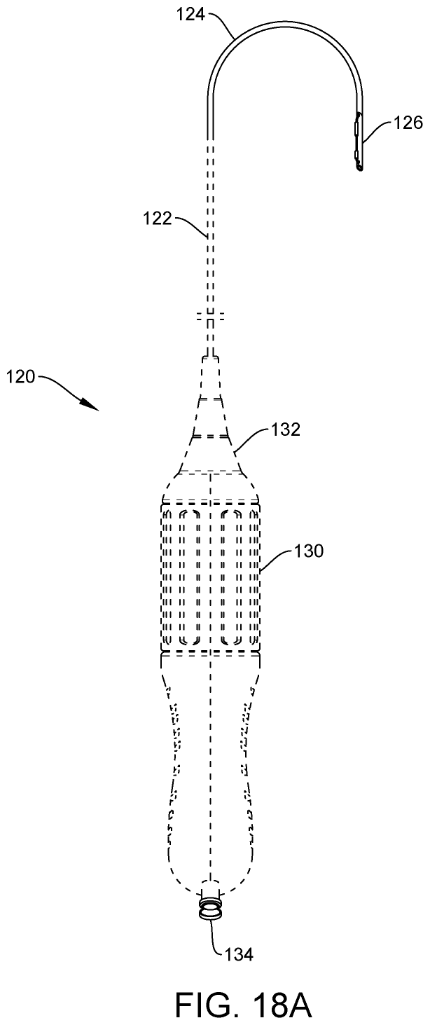

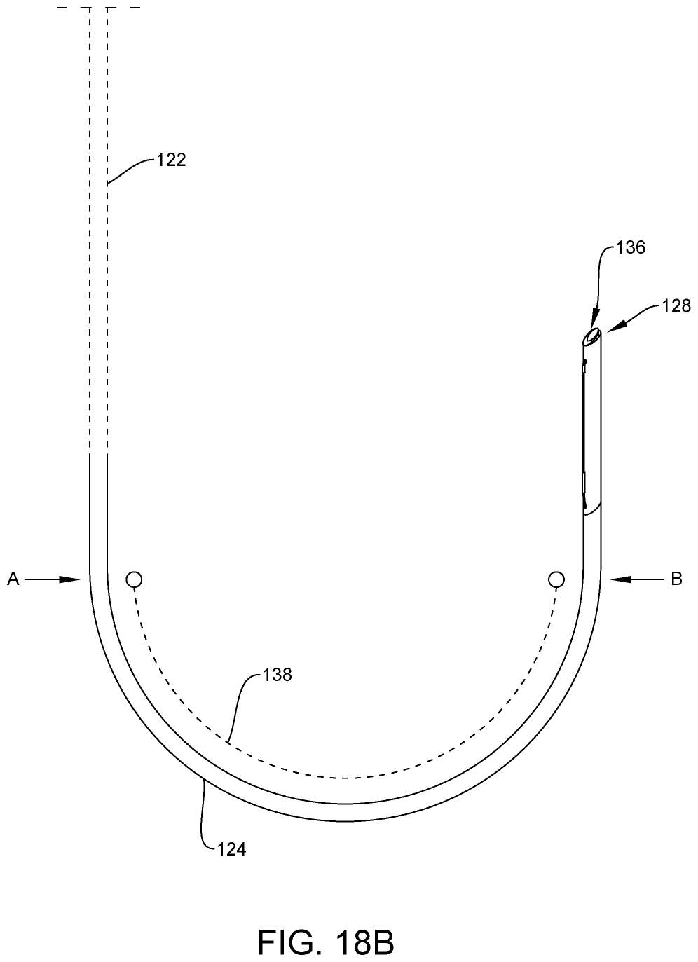

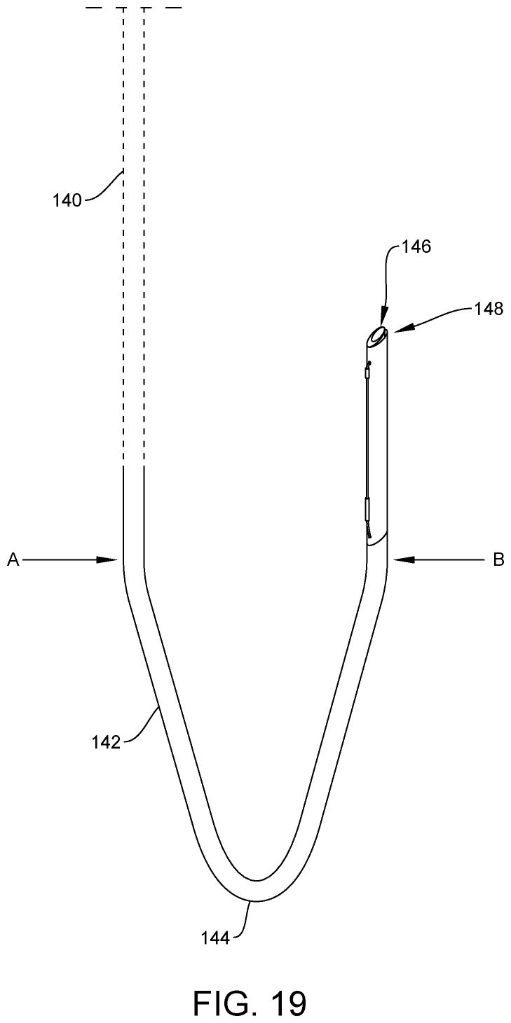

FIG. 17 shows an example of a cardiac pacemaker device 100 that could be implanted using the catheter device 10. The pacemaker device 100 comprises an electrical pulse generator 104 and an electrode lead. The electrode lead comprises a lead wire 102 and an electrode 110 at the distal end. Also at the distal end are a ring cap 106 and tines 108 for anchoring within the myocardium.

FIGS. 18A and 18B shows another example of a catheter device 120 of this invention. FIG. 18A shows the catheter device 120 comprising a handle assembly 130, a cone-shaped hood 132, and a port 134 at the back for inserting a puncture tool. Also shown is a flexible main tube 122 which has a pre-formed C-shape curve segment 124 and a bendable segment 126. FIG. 18B shows a close-up view of the distal portion of the main tube 122. Shown is the pre-formed C-shape curve segment 124, which is defined according to points A and B as described above. Dashed line 138 represents the length of the C-shape curve segment 124 (i.e. the distance from points A to B when overlaid on C-shape curve segment 124). At the distal tip 128 of the main tube 122, there is an exit hole 136 where the puncture tool (not shown) exits.

FIG. 19 shows another example of how the distal portion of the flexible main tube 140 could be shaped. Here, instead of a C-shape curve, the main tube 140 has a V-shaped curve 142. This V-shaped curve has an apex 144. This V-shaped curve 142 could be defined according to the same dimensional measurements as described above for the C-shape curve. Also shown is the distal tip 148 of the main tube 140 and an exit hole 146 where the puncture tool (not shown) exits.

### EXPERIMENTAL

Prototypes of the experimental catheter device were tested in 10 swine animals. The procedure was performed by inserting a sheath into the entry vein (jugular, subclavian, or femoral vein). A first guidewire was inserted through this sheath and maneuvered into the coronary sinus. Along this guidewire path, a balloon-tipped guiding catheter was introduced into the heart and into the coronary sinus. The balloon was engaged with the coronary sinus to form a seal and radiopaque contrast agent was injected under pressure to obtain an x-ray fluoroscopic image (i.e. pressurized venogram). This pressurized venogram confirmed whether or not a septal perforator vein was present in the subject swing animal.

The first guidewire was exchanged with a thinner second guidewire for introducing the experimental catheter device. This thinner guidewire was advanced into the great cardiac vein. The catheter device was introduced along this guidewire into the great cardiac vein and positioned at a suitable place and vector direction to engage the septal myocardium. At this point, the distal end of the catheter device was bent at an angle towards the septal myocardium

The second guidewire was exchanged with a stiffer third guidewire to serve as a puncture wire. The puncture wire was pushed forward to puncture directly through the wall of the great cardiac vein and enter the myocardium towards the septum. The amount of force needed to push the puncture wire through the vein wall and into the myocardium was measured using a push-pull gauge. A force of 0.45 N (newtons) was required to penetrate through the vein wall. Once inside the myocardium, a lesser force of 0.04 N was needed to push through the myocardium tissue.

With guidance by x-ray fluoroscopy, the puncture wire was further advanced to the target site within the interventricular septum. Ultrasound echocardiography and x-ray fluoroscopy confirmed that the puncture wire was in the desired location. This puncture procedure was successfully performed in all 10 of the swine animals.

After the procedure experiments were completed, the hearts were harvested for visual pathologic and histopathologic examination. On visual examination, there was no significant systematic damage to the blood vessels or myocardial walls. There was a hematoma around the puncture site, but there was no spread of the hematoma or severe damage to other areas. There was no hemorrhage in the epicardium where the guidewire entered the myocardium. With microscopic histopathology examination, no myocardial damage was seen. There were some hemorrhagic foci in the myocardium and epicardium. But there was no ischemic damage or necrosis.

The descriptions and examples given herein are intended merely to illustrate the invention and are not intended to be limiting. Each of the disclosed aspects and embodiments of the invention may be considered individually or in combination with other aspects, embodiments, and variations of the invention. In addition, unless otherwise specified, the steps of the methods of the invention are not confined to any particular order of performance. Modifications of the disclosed embodiments incorporating the spirit and substance of the invention may occur to persons skilled in the art, and such modifications are within the scope of the invention.

Any use of the word “or” herein is intended to be inclusive and is equivalent to the expression “and/or,” unless the context clearly dictates otherwise. As such, for example, the expression “A or B” means A, or B, or both A and B. Similarly, for example, the expression “A, B, or C” means A, or B, or C, or any combination thereof.

1. A method of creating a transvenous access pathway into an interventricular septum of a patient's heart, comprising:
having a catheter device comprising a main tube; wherein the main tube comprises a distal end segment, a distal tip, and a lumen; wherein the catheter device further comprises a puncture tool that travels through the lumen;
inserting the main tube into an entry vein;
advancing the catheter device so that the main tube travels into a coronary sinus and is positioned at a target site inside a coronary vein;
bending the distal end segment of the main tube so that the distal tip points at an angle towards a wall of the coronary vein;
pushing the puncture tool out of the main tube to puncture through the wall of the coronary vein;
advancing the puncture tool through myocardium towards the interventricular septum to create an entry passageway to the interventricular septum.

2. The method of claim 1, wherein the catheter device further comprises a steering wire that is fixed at the distal end segment of the main tube;
wherein the step of bending the distal end segment of the main tube comprises pushing or pulling the steering wire.

3. The method of claim 2, wherein steering wire is fixed at a point that is within 1.0 cm of the distal tip of the main tube.

4. The method of claim 2, wherein the catheter device further comprises a handle assembly, which comprises a steering actuator;
wherein the steering wire is coupled to the steering actuator which acts to push or pull the steering wire.

5. The method of claim 4, wherein the lumen is a first lumen for the puncture tool; wherein the main tube further comprises a second lumen that is separate from the first lumen; wherein the steering wire travels through the separate second lumen of main tube.

6. The method of claim 4, wherein the main tube comprises an exit hole out of the second lumen, and wherein the steering wire exits the second lumen out of the exit hole.

7. The method of claim 4, wherein the steering actuator comprises a slider and the steering wire is fixed to the slider; wherein the method further comprises:
moving the slider back and forth to push or pull the steering wire.

8. The method of claim 1, wherein the bend angle is at least 45° relative to the longitudinal axis of the main tube.

9. The method of claim 1, wherein the distal end segment of the main tube comprises three or more radiopaque markers, and the method further comprises:
observing the alignment of the radiopaque markers under x-ray fluoroscopy to determine the amount of bending at the distal end segment of the main tube.

10. The method of claim 1, further comprising applying a force of 0.205-1.25 N (newtons) to the puncture tool in advancing the puncture tool through the vein wall.

11. The method of claim 10, wherein the coronary vein is a great cardiac vein.

12. The method of claim 1, wherein the coronary vein is a septal perforating vein, and the method further comprising applying a force of less than 0.20 N (newtons) in advancing the puncture tool through the vein wall.

13. The method of claim 1, further comprising performing a pressurized venogram of the coronary vein.

14. The method of claim 1, wherein the main tube further comprises a pre-formed C-shape curve segment; wherein the C-shape curve segment follows the natural curve path of the coronary sinus.

15. The method of claim 1, wherein the main tube comprises a coil element at the distal end segment, wherein the bending occurs at the coil element.

16. The method of claim 1, wherein the access pathway is for positioning an electrode lead inside the interventricular septum; and wherein the method further comprises:
withdrawing the puncture tool out of the catheter device;
inserting the electrode lead through the lumen so that it travels into the coronary sinus and through the coronary vein;
advancing the electrode lead through the puncture hole in the vein wall and into the entry passageway of the interventricular septum.

17. The method of claim 16, wherein the electrode lead is a pacing lead and the method further comprises activating pacing stimulation to cause pacing of the heart.

18. The method of claim 16, the electrode lead is a radiofrequency (RF) ablation lead and the method further activating the electrode to ablate heart tissue.

19. The method of claim 1, wherein the access pathway is for a mitral loop cerclage wire in performing a mitral valve cerclage procedure; and wherein the method further comprises:
withdrawing the puncture tool out of the catheter device;
inserting the cerclage wire through the lumen so that it travels into the coronary sinus and through the coronary vein;
advancing the cerclage wire through the puncture hole in the vein wall and into the entry passageway of the interventricular septum.

20. The method of claim 1, further comprising using an opposing wall of the coronary vein as a buttress for applying pushing force to the puncture tool.

21. A catheter device comprising:
a main tube comprising a distal end segment, a distal tip, a lumen, and an exit hole out of the lumen; wherein the main tube comprises a pre-formed C-shape curve segment that extends from a proximal point A to a distal point B on the main tube, wherein point A is located within an interval that is 6.0-15 cm of the distal tip of the main tube and point B is located within an interval that is 0.4-4.0 cm from the distal tip of the main tube; wherein point B is located distal to point A and the length of the C-shape curve segment distance from point A to point B is in the range of 5-20 cm long;
a steering wire that travels through the lumen and is fixed at the distal end segment of the main tube at a point that is within 1.0 cm of the distal tip of the main tube; and wherein the steering wire exits the lumen out of the exit hole of the main tube;
a handle body comprising a steering actuator, wherein the steering wire is coupled to the steering actuator which acts to push or pull the steering wire.

22. The device of claim 21, wherein the steering actuator comprises a slider and the steering wire is fixed to the slider.

23. A septal puncture assembly comprising the device of claim 21, wherein the lumen is a first lumen and the main tube further comprises a second lumen; and wherein the septal puncture assembly further comprises a puncture tool that travels through the second lumen.
